(19) United States
(12) Patent Application Publication
(10) Pub. No.: US 2011/0152227 A1
(43) Pub. Date: Jun. 23, 2011

(54) DEUTERIUM LABELLED DERIVATIVES OF 3-(2-HYDROXY-5-METHYPHENYL)-N,N-DIISOPROPYL-3-PHENYLPROPYLAMINE AND METHODS OF USE THEREOF

(75) Inventor: Julie F. Liu, Lexington, MA (US)

(73) Assignee: CONCERT PHARMACEUTICALS INC., Lexington, MA (US)

(21) Appl. No.: 12/937,262

(22) PCT Filed: Apr. 9, 2009
(86) PCT No.: PCT/US09/40126

§ 371 Date: Jan. 26, 2011

Related U.S. Application Data
(60) Provisional application No. 61/043,729, filed on Apr. 9, 2008.

(51) Int. Cl. A61K 31/56; C07C 211/01; A61K 31/137; A61K 31/403; A61K 31/18; C07C 69/67; A61K 31/222; A61P 35/00; A61P 13/10; A61P 25/28; A61P 9/00; A61P 11/00; A61P 15/10

(52) U.S. Cl. 514/171; 564/316; 514/648; 514/412; 514/603; 560/250; 514/546

(57) ABSTRACT

This invention relates to novel derivatives of tolterodine, 5-hydroxymethyl tolterodine, fesoterodine and pharmaceutically acceptable salts thereof. This invention also provides compositions comprising a compound of this invention and the use of such compositions in methods of treating diseases and conditions that are beneficially treated by muscarinic receptor antagonists.

### CROSS-REFERENCE TO RELATED APPLICATIONS

This application claims the benefit of U.S. Provisional Patent Application No. 61/043,729, filed Apr. 9, 2008, the contents of which are incorporated herein by reference in their entirety.

This invention relates to novel derivatives of tolterodine, 5-hydroxymethyl tolterodine, and fesoterodine, pharmaceutically acceptable salts, solvates, and hydrates thereof. This invention also provides compositions comprising a compound of this invention and the use of such compositions in methods of treating diseases and conditions that are beneficially treated by muscarinic receptor antagonists.

Tolterodine is 3-(2-hydroxy-5-methylphenyl)-N,N-diisopropyl-3-phenylpropylamine and is sold as its 1:1 salt with L-tartaric acid under the name Detrol®. It is a potent, competitive muscarinic receptor antagonist, which is useful, e.g., for treating adults who have overactive bladder.

Detrol is approved for treatment of patients with overactive bladder with symptoms of frequency, urgency, urge incontinence, or any combination of these symptoms. Tolterodine is also in clinical trials studying the effect of the compound on memory and cognition in the elderly.

After oral administration tolterodine is metabolized in the liver, resulting in the formation of 5-hydroxymethyl tolterodine, a major pharmacologically active metabolite. The 5-hydroxymethyl metabolite, which exhibits an antimuscarinic activity similar to that of tolterodine, contributes significantly to the therapeutic effect.

Fesoterodine is a prodrug of the 5-hydroxymethyl metabolite and is marketed as the fumaric acid salt under the names Toviaz®, 2-methylpropionic acid 243-(N,N-diisopropylamino)-[(R)-phenylpropyl]-4-(hydroxymethyl)phenyl ester fumarate, and isobutyric acid 2-[3-(diisopropylamino)-1(R)-phenylpropyl]-4-(hydroxymethyl)phenyl ester fumarate. Fesoterodine has been approved in the European Union for the treatment of urge incontinence and overactive bladder and is in clinical trials in the U.S. as an antimuscarinic.

Despite the beneficial activities of tolterodine, 5-hydroxymethyl tolterodine, and fesoterodine, there is a continuing need for new compounds to treat the aforementioned diseases and conditions.

### BRIEF DESCRIPTION OF THE DRAWING

FIG. 1 depicts the time course of metabolism of various compounds of the invention in human liver microsomes as compared to tolterodine.

### DEFINITIONS

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein).

By “disease” is meant any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of tolterodine, 5-hydroxymethyl tolterodine, or fesoterodine will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al, Seikagaku 1994, 66:15; Gannes L Z et al., Comp Biochem Physiol Mol Integr Physiol 1998, 119:725. In a compound of this invention, when a particular position is designated as having deuterium, it is understood that the abundance of deuterium at that position is substantially greater than the natural abundance of deuterium, which is 0.015%. A position designated as “D” or deuterium typically has deuterium at that position at an abundance of at least 3000 times greater than the natural abundance of deuterium (i.e., at least 45% deuterium incorporation).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

The term “isotopologue” refers to a species that differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 55% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts, solvates or hydrates of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention (e.g., compounds of Formula I, Formula II, etc.), contain an asymmetric carbon atom. As such, compounds of this invention can exist as either individual enantiomers (e.g., one of (S) or (R)), or mixtures of the two enantiomers. The (R) compound has greater activity and is preferred. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium.

“Stereoisomer” refers to both enantiomers and diastereomers.

“Tert”, “^{t}”, and “t-” each refer to tertiary.

“US” refers to the United States of America.

“FDA” refers to Food and Drug Administration.

“E.U.” refers to the European Union.

Throughout this specification, reference to “each R” includes, independently, any “R” group (e.g., R¹, R², R³, R⁴, and R⁵) where applicable. Unless otherwise indicated, when a variable is referred to generally, it is meant to include all specific embodiments of that particular variable.

### Therapeutic Compounds

The present invention provides a compound of Formula I:

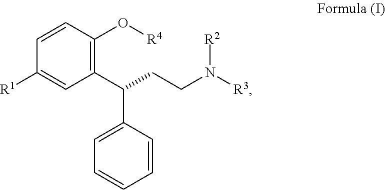

or a pharmaceutically acceptable salt, solvate, or hydrate thereof, wherein:

R¹ is CD₃, CHD₂, CH₂D, CH₃, CD₂OH, CHDOH, or CH₂OH;

R² and R³ are each, independently, an isopropyl group bearing zero to seven deuterium atoms;

R⁴ is H or C(O)—C₁-C₆ alkyl; and

at least one of R¹, R², or R³ comprises a deuterium atom.

In certain embodiments, R¹ is selected from CH₃, CD₃, CD₂OH, or CH₂OH.

In certain embodiment, R² and R³ are the same.

In other embodiments, R² and R³ are independently selected from —CH(CD₃)₂, —CD(CD₃)₂, —CH(CH₃)₂, and —CD(CH₃)₂.

In certain embodiments, R⁴ is H.

In other embodiments, R⁴ is C(O)—CH(CH₃)₂.

In yet another embodiment, the compound is selected from any one of the compounds set forth in Table 1 (below):

TABLE 1
Exemplary Embodiments of Formula I

| Compound | R¹ | R² | R³ | R⁴ |
|---|---|---|---|---|
| 100 | CD₃ | —CD(CD₃)₂ | —CD(CD₃)₂ | H |
| 101 | CD₃ | —CD(CD₃)₂ | —CD(CH₃)₂ | H |
| 102 | CD₃ | —CD(CH₃)₂ | —CD(CH₃)₂ | H |
| 103 | CD₂OH | —CD(CD₃)₂ | —CD(CD₃)₂ | H |
| 104 | CD₂OH | —CD(CD₃)₂ | —CD(CH₃)₂ | H |
| 105 | CD₂OH | —CD(CH₃)₂ | —CD(CH₃)₂ | H |
| 106 | CH₃ | —CD(CD₃)₂ | —CD(CD₃)₂ | H |
| 107 | CH₃ | —CD(CD₃)₂ | —CD(CH₃)₂ | H |
| 108 | CH₃ | —CD(CH₃)₂ | —CD(CH₃)₂ | H |
| 109 | CH₂OH | —CD(CD₃)₂ | —CD(CD₃)₂ | H |
| 110 | CH₂OH | —CD(CD₃)₂ | —CD(CH₃)₂ | H |
| 111 | CH₂OH | —CD(CH₃)₂ | —CD(CH₃)₂ | H |
| 112 | CD₃ | —CH(CH₃)₂ | —CH(CH₃)₂ | H |
| 113 | CD₂OH | —CH(CH₃)₂ | —CH(CH₃)₂ | H |
| 114 | CD₃ | —CH(CD₃)₂ | —CH(CD₃)₂ | H |
| 115 | CD₂OH | —CH(CD₃)₂ | —CH(CD₃)₂ | H |
| 116 | CD₂OH | —CH(CH₃)₂ | —CH(CH₃)₂ | —C(O)—CH(CH₃)₂ |
| 117 | CH₂OH | —CD(CD₃)₂ | —CD(CD₃)₂ | —C(O)—CH(CH₃)₂ |
| 118 | CD₂OH | —CD(CD₃)₂ | —CD(CD₃)₂ | —C(O)—CH(CH₃)₂ |

In yet another embodiment, the invention provides a compound of Formula II:

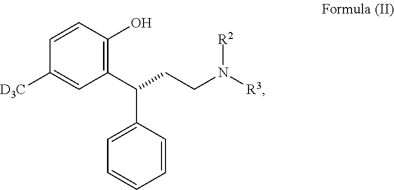

or a pharmaceutically acceptable salt thereof, wherein:

R² and R³ are each, independently, an isopropyl group bearing zero to seven deuterium atoms.

In one embodiment of Formula II, each of R² and R³ are independently selected from —CD(CD₃)₂ and —CH(CH₃)₂. In a more specific aspect, the compound of Formula II is selected from Compound 100 and Compound 112.

In still another embodiment, the invention provides a compound of Formula III:

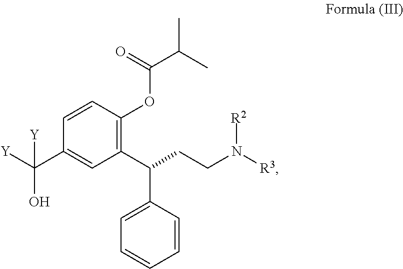

or a pharmaceutically acceptable salt thereof, wherein:

R² and R³ are each, independently, an isopropyl group bearing zero to seven deuterium atoms; and both Y are hydrogen or deuterium,

wherein when both Y are hydrogen, at least one of R² or R³ comprises a deuterium atom.

In one embodiment of Formula III, each of R² and R³ are independently selected from —CD(CD₃)₂ and —CH(CH₃)₂. In a more specific aspect, the compound of Formula III is selected from Compound 116, Compound 117 or Compound 118.

In another set of embodiments, any atom not otherwise designated as deuterium in any of the preceding embodiments is present at its natural isotopic abundance.

The synthesis of compounds of Formula I, Formula II, etc., can be readily achieved by synthetic chemists of ordinary skill Relevant procedures and intermediates are disclosed, for instance in U.S. Pat. Nos. 7,119,212, 7,005,449, 5,382,600, 5,922,914, 5,686,464, and 5,559,269; PCT Publication Nos. WO2005/006143, WO2005/012227, WO/2004/078700, WO2001/049649, WO2003/014060, and WO2006/074479; Andersson, P G et al, J Org Chem 1998, 63(22):8067; and EP Patent Publication EP0957073.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

A convenient method for synthesizing compounds of Formula I, Formula II, etc., is depicted in Scheme 1.

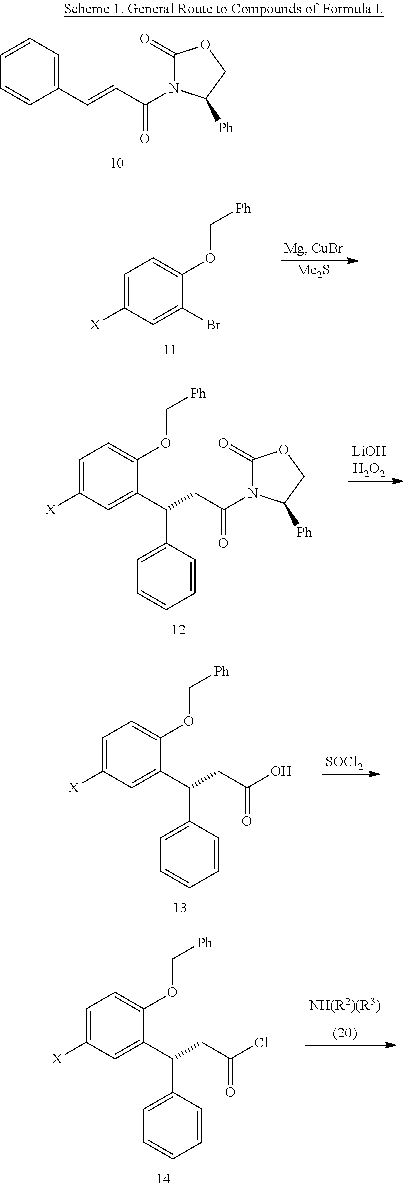

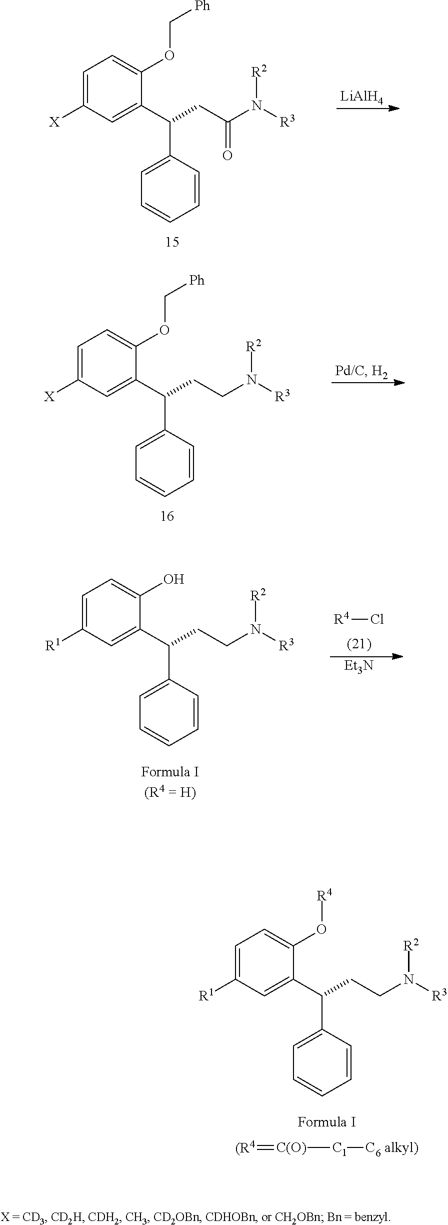

As shown in Scheme 1, the preparation of compounds of Formula I begins with the regioselective addition of an appropriately deuterated aryl bromide 11 to known 4(R)-phenyl-3-[3-phenyl-2(E)-propenoyl]oxazolidin-2-one (10) using Mg/CuBr.dimethylsulfide to produce adduct 12. Hydrolysis of 12 with LiOH/H₂O₂ in THF/water provides the carboxylic acid 13. The reaction of 13 with SOCl₂/pyridine in benzene yields the acid chloride 14, which is treated with an appropriately deuterated amine 20 (such as commercially available diisopropyl amine-d_{14 [}98 atom % D]) to afford the corresponding amide 15. The reduction of 15 with LiAlH₄ in ether gives the tertiary amine 16, which is debenzylated by hydrogenation with H₂ over Pd/C in methanol to yield a compound of Formula I, wherein R⁴ is H. The phenolic hydroxyl of 17 may be acylated with an acyl chloride 21 in the presence of Et₃N to afford a compound of Formula I, wherein R⁴ is —C(O)—C₁-C₆ alkyl. Reference: general procedures in Andersson, P G et al., “Asymmetric total synthesis of (+)-tolterodine, a new muscarinic receptor antagonist, via copper-assisted asymmetric conjugate addition of aryl Grignard reagents to 3-phenyl-prop-2-enoyl-oxazolidinones,” J Org Chem, 1998, 63(22): 8067; and EP Patent Publication EP957073.

Exemplary methods for preparing an appropriately deuterated aryl bromide 11 are shown in Schemes 2A and 2B, below.

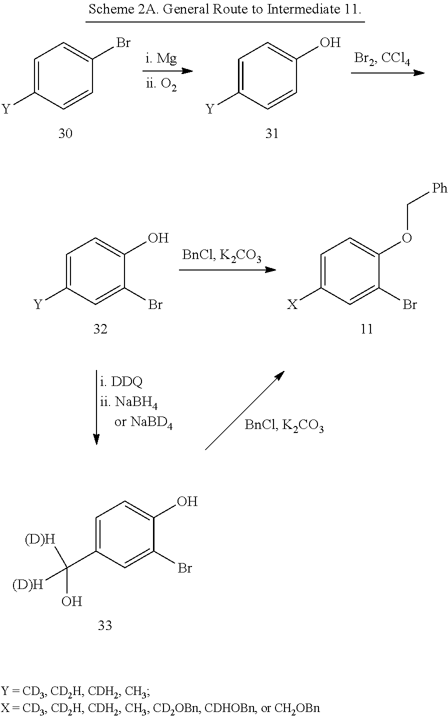

As depicted in Scheme 2A (following the general procedures in Aki, et al, J Phys Chem A 2002, 106(14):3436-3444), the desired aryl bromide 30 is converted to the phenol 31 via Mg and O₂ and then brominated with Br₂ to provide the bromophenol 32. The bromophenol 32 may either be benzyl-protected directly via benzyl chloride to yield compound II, or may be oxidized with DDQ then reduced to the appropriately deuterated alcohol 33 using NaBH₄ or NaBD₄ (98 atom % D). The diol 33 may then be benzyl-protected with benzyl chloride to afford compound 11.

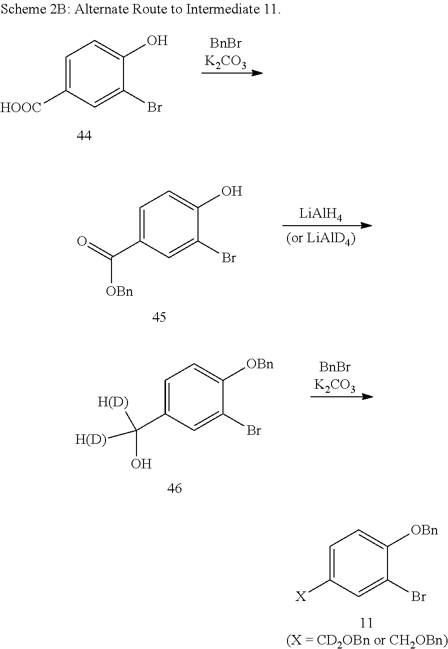

As depicted in Scheme 2B, commercially-available 3-bromo-4-hydroxybenzoic acid is treated with benzyl bromide and potassium carbonate to afford ester 45. Reduction of the ester with either LiAlH₄ or LiAlD₄ (99 atom % D) provides alcohol 46. Treatment with benzyl bromide and potassium carbonate provides compound II wherein X is either CD₂OBn or CH₂OBn.

Methods for making an appropriately deuterated amine 20 are shown in Schemes 3A and 3B, below.

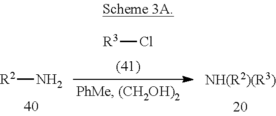

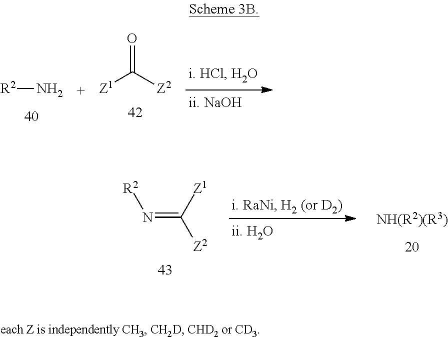

As shown in Scheme 3A, an appropriately deuterated amine 40 is alkylated with an appropriately deuterated 2-chloropropane 41 (or any other appropriately deuterated 2-halopropane) to provide desired amine 20 (see, e.g., a similar procedure in Zhang, C, et al, Zhongguo Yaowu Huaxue Zazhi 2004, 14(3):161-164).

Alternately, as shown in scheme 3B, amine 40 is condensed with an appropriately deuterated 2-ketopropane 42 to yield imine 43, which is reduced with appropriately-deuterated Raney Nickel and hydrogen (or deuterium) gas to deliver amine 20. See, e.g., Bunnelle, W H, et al., Synthesis, 1997, 4: 439-442; and Norton, D G et al., J Org Chem 1954, 19: 1054-66.

Additional methods of synthesizing compounds of Formula I, Formula II, etc., and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free compositions comprising an effective amount of a compound of Formula I, Formula II, etc. (e.g., including any of the formulae herein), or a pharmaceutically acceptable salt, solvate, or hydrate of said compound; and an acceptable carrier. In one embodiment, the composition is pyrogen-free. In another embodiment, the composition is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention. For example, the compounds of Formula I may be formulated for topical or transdermal compositions in a manner similar to that known in the art for formulating tolterodine as described in, for example, PCT publication WO2005107812.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. For example, the compounds of Formula I, Formula II, etc., may be formulated for controlled release in a manner similar to that known in the art for formulating tolterodine as described in, for example, WO2007011131; WO2007022255; WO2006021425; WO2005105036; WO2004105735; WO2001034139; WO2000027364.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible through surgery or removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as tolterodine. Such agents include those indicated as being useful in combination with tolterodine, including but not limited to, an alpha-adrenergic receptor antagonist, such as those described in PCT Patent Publication WO2001/021167 and WO/2007/010509; a bicifadine compound such as those described in PCT Patent Publication WO2006/102029; a statin, such as those described in PCT Patent Publication WO2006/008437; a dehydroepiandrosterone (DHEA) congener, such as those described in PCT Patent Publication WO2006/007312; a alpha-2-delta subunit calcium channel modifier, such as a GABA analog and others described in PCT Patent Publication WO2004/084879; a selective serotonin reuptake inhibitor (SSRI) or a selective norepinephrine uptake inhibitor, such as fluoxetine, paroxetine and others described in PCT Patent Publications WO2004/019892 and WO2001/062236; an androgen, an estrogen or an estrogen agonist, such as those described in PCT Patent Publications WO2004/043429, WO2003/039553 and WO2003039523; an EGF receptor antagonist, such as those described in PCT Patent Publication WO2003/039524; a 5HT1a receptor modifier such as those described in PCT Patent Publication WO2003/026564; a thienopyranecarboxamide derivative, such as those described in PCT Patent Publication WO2001/009140; a 5a reductase inhibitor, such as those described in PCT Patent Publication WO2001/021167, and those second agents described in U.S. Patent Publication Nos. 2006-205682 and 2006-160887, in PCT Patent Publication Nos. WO2006/079625, WO2006/015970, WO2005/092342, WO2005/092341, and WO2001/062236, and in Japanese Patent Publications JP2003-055261 and JP2005-015394.

In one embodiment, the second therapeutic agent is an agent useful in the treatment or prevention of a disease or condition selected from unstable or overactive bladder, incontinence, infection, lower urinary tract disorders, memory or cognition impairment, heart failure, pneumonia (including alveolar pneumonia), benign prostatic hyperplasia, prostatic hypertrophy, respiratory disorders, asthma, female sexual dysfunction, or cystitis.

In one embodiment, the second therapeutic agent is tamsulosin,

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from 0.1 mg/day to 50 mg/day for an adult human patient. In another embodiment, an effective amount of a compound of this invention can range from 1 mg/day to 10 mg/day for an adult human patient. In yet another embodiment, an effective amount can range from 2-6 mg/day for an adult human patient. In still another embodiment, an effective amount is about 4 mg per day for an adult human patient.

Effective doses will also vary, as recognized by those skilled in the art, depending on the diseases treated, the severity of the disease, the route of administration, the size, sex, age and general health condition of the patient, excipient usage, the possibility of co-usage with other therapeutic treatments such as use of other agents and the judgment of the treating physician. For example, guidance for selecting an effective dose can be determined by reference to the prescribing information for tolterodine.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating the activity of a cholinergic muscarinic receptor in a cell, comprising contacting a cell with one or more compounds of Formula I, Formula II, etc., herein.

According to another embodiment, the invention provides a method of treating a subject suffering from, or susceptible to, a disease that is beneficially treated by tolterodine comprising the step of administering to said subject in need thereof an effective amount of a compound or a composition of this invention. Such diseases are well known in the art and are disclosed in, but not limited to the following patents and/or published applications: U.S. Patent Publication Nos. 2006-205682 and 2006-160887, and PCT Patent Publication Nos. WO2006/079625, WO2006/015970, WO2005/092342, WO2005/092341, WO2001/062236, WO2003/039553, WO2003/039524, and WO2003/026564, and Japanese Patent Publications JP2003-055261 and JP2005-015394.

In another embodiment, the method of this invention is used to treat a subject suffering from or susceptible to a disease or condition selected from overactive bladder, incontinence, lower urinary tract infection, asthma, benign prostatic hypertrophy and memory and cognition impairment. In certain embodiments, the method of this invention is used to treat a subject suffering from or susceptible to a disease or condition selected from overactive bladder and incontinence. Methods delineated herein also include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with tolterodine. Examples of such agents and the conditions and diseases for which each may be used in conjunction with a compound of this invention include (1) an alpha-adrenergic receptor antagonist, such as tamsulosin and those described in PCT Patent Publication WO2001/021167 and WO/2007/010509; a bicifadine compound such as those described in PCT Patent Publication WO2006/102029; an alpha-2-delta subunit calcium channel modifier, such as a GABA analog and others described in PCT Patent Publication WO2004/084879; a selective serotonin reuptake inhibitor (SSRI) or a selective norepinephrine uptake inhibitor, such as fluoxetine, paroxetine and others described in PCT Patent Publications WO2004/019892 and WO2001/062236; a 5HT1a receptor modifier such as those described in PCT Patent Publication WO2003/026564; a thienopyranecarboxamide derivative, such as those described in PCT Patent Publication WO2001/009140; or a 5a reductase inhibitor, such as those described in PCT Patent Publication WO2001/021167 each for the treatment of lower urinary tract disorders (including incontinence, unstable or overactive bladder, and other urinary disorders); (2) a dehydroepiandrosterone (DHEA) congener, such as those described in PCT Patent Publication WO2006/007312 for use in treating inflammation; (3) a statin, such as those described in PCT Patent Publication WO2006/008437 for use in treating a respiratory disease; (4) an androgen, an estrogen or an estrogen agonist, such as those described in PCT Patent Publications WO2004/043429, WO2003/039553 and WO2003039523 for treating female sexual dysfunction; (5) an EGF receptor antagonist, such as those described in PCT Patent Publication WO2003/039524; or a thienopyranecarboxamide derivative, such as those described in PCT Patent Publication WO2001/009140 for treating benign prostatic hyperplasia or prostatic hypertrophy; as well as those described in U.S. Patent Publication Nos. 2006-205682 and 2006-160887, in PCT Patent Publication Nos. WO2006/079625, WO2006/015970, WO2005/092342, WO2005/092341, and WO2001/062236, and in Japanese Patent Publications JP2003-055261 and JP2005-015394.

In particular, the combination therapies of this invention include treatment of the following conditions by administering a compound of Formula I and a second therapeutic agent: overactive bladder and incontinence.

According to one embodiment, a compound of Formula I is co-administered with tamsulosin to treat a lower urinary tract disorder.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I, Formula II, etc., alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of Formula I, Formula II, etc., for use in the treatment or prevention in a subject of a disease, disorder or symptom thereof delineated herein.

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

### Diagnostic Methods and Kits

The compounds and compositions of this invention are also useful as reagents in methods for determining the concentration of tolterodine, 5-hydroxymethyl tolterodine, or fesoterodine in solution or biological sample such as plasma, examining the metabolism of tolterodine, 5-hydroxymethyl tolterodine, or fesoterodine and other analytical studies.

According to one embodiment, the invention provides a method of determining the concentration, in a solution or a biological sample, of tolterodine, 5-hydroxymethyl tolterodine, or fesoterodine, comprising the steps of:
a) adding a known concentration of a compound of Formula Ito the solution of biological sample;
b) subjecting the solution or biological sample to a measuring device that distinguishes tolterodine, 5-hydroxymethyl tolterodine, or fesoterodine from a compound of Formula I;
c) calibrating the measuring device to correlate the detected quantity of the compound of Formula I with the known concentration of the compound of Formula I added to the biological sample or solution; and
d) measuring the quantity of tolterodine, 5-hydroxymethyl tolterodine, or fesoterodine in the biological sample with said calibrated measuring device; and
e) determining the concentration of tolterodine, 5-hydroxymethyl tolterodine, or fesoterodine in the solution of sample using the correlation between detected quantity and concentration obtained for a compound of Formula I.

Measuring devices that can distinguish tolterodine, 5-hydroxymethyl tolterodine, or fesoterodine from the corresponding compound of Formula I include any measuring device that can distinguish between two compounds that differ from one another only in isotopic abundance. Exemplary measuring devices include a mass spectrometer, NMR spectrometer, or IR spectrometer.

In another embodiment, the invention provides a method of evaluating the metabolic stability of a compound of Formula I comprising the steps of contacting the compound of Formula I with a metabolizing enzyme source for a period of time and comparing the amount of the compound of Formula I with the metabolic products of the compound of Formula I after the period of time.

In a related embodiment, the invention provides a method of evaluating the metabolic stability of a compound of Formula I in a patient following administration of the compound of Formula I. This method comprises the steps of obtaining a serum, urine or feces sample from the patient at a period of time following the administration of the compound of Formula I to the subject; and comparing the amount of the compound of Formula I with the metabolic products of the compound of Formula I in the serum, urine or feces sample.

The present invention also provides kits for use to treat overactive bladder and incontinence. These kits comprise (a) a pharmaceutical composition comprising a compound of Formula I or a salt, hydrate, or solvate thereof, wherein said pharmaceutical composition is in a container; and (b) instructions describing a method of using the pharmaceutical composition to treat overactive bladder and incontinence.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In on embodiment, the container is a blister pack.

The kit may additionally comprise a memory aid of the type containing information and/or instructions for the physician, pharmacist or subject. Such memory aids include numbers printed on each chamber or division containing a dosage that corresponds with the days of the regimen which the tablets or capsules so specified should be ingested, or days of the week printed on each chamber or division, or a card which contains the same type of information. For single dose dispensers, memory aids further include a mechanical counter which indicates the number of daily doses that have been dispensed and a battery-powered micro-chip memory coupled with a liquid crystal readout and/or audible reminder signal which, for example, reads out the date that the last daily dose has been taken and/or reminds one when the next dose is to be taken. Other memory aids useful in such kits are a calendar printed on a card, as well as other variations that will be readily apparent.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

### EXAMPLES

### Example 1

### Synthesis of Various Intermediates 11

### A. 1-(Benzyloxy)-2-bromo-4-methylbenzene (11a, X=CH₃)

Intermediate 11a was prepared following the general procedure of Aki et al., J Phys Chem, 2002, 106(14): 3436-3444.

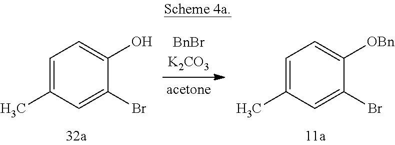

1-(Benzyloxy)-2-bromo-4-methylbenzene (11a, X=CH₃). To a solution of 2-bromo-4-methylphenol 32a (5 g, 26.7 mmol) in acetone (60 mL) was added K₂CO₃ (11 g, 80 mmol) followed by benzyl bromide (3.8 mL, 32 mmol). The mixture was heated to reflux for 4 hours, then was cooled to room temperature. Solid was removed via filtration and the filtrate was concentrated in vacuo. The residue was purified by column chromatography (5% EtOAc/heptane) to give 6.7 g of 11a as a white semi-solid (yield 90%).

### B. 1-(Benzyloxy)-2-bromo-4-(methyl-d₃)benzene (11b, X=CD₃)

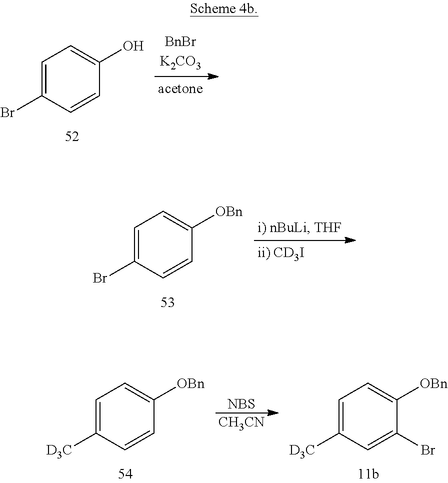

Step 1. 1-(Benzyloxy)-4-bromobenzene (53). To a solution of 4-bromophenol (52) (5 g, 29 mmol) in acetone (60 mL) was added K₂CO₃ (12.4 g, 90 mmol) followed by benzyl bromide (3.8 mL, 32 mmol). The mixture was heated to reflux for 4 hours, then was cooled to room temperature. Solid was removed via filtration and the filtrate was concentrated in vacuo. The residue was purified by column chromatography (5% EtOAc/heptane) to give 6.9 g of 53 as a white solid (yield 90%).

Step 2. 1-(Benzyloxy)-4-(methyl-d₃)benzene (54). A solution of 1-(benzyloxy)-4-bromobenzene (53) (5 g, 19 mmol) in THF (anhydrous, 40 mL) was cooled to −78° C., then n-BuLi (9.1 mL in 2.5M hexane, 22.8 mmol) was added dropwise. The reaction mixture was stirred for 30 minutes, then CD₃I [Cambridge Isotope, 99 atom % D] (1.4 mL, 22.8 mmol) was added. The mixture was stirred for 1 hour then was quenched by the addition of aqueous NH₄Cl (satd). The mixture was extracted with EtOAc (100 mL), and the organic layer was washed with brine, dried over Na₂SO₄, filtered, concentrated in vacuo, and purified by column chromatography (5% EtOAc/heptane) to give 3.05 g of 54 as a yellow oil (yield 80%).

Step 3. 1-(Benzyloxy)-2-bromo-4-(methyl-d₃)benzene (11b, X=CD₃). To a solution of 54 (3.0 g, 15 mmol) in 50 mL acetonitrile was added N-bromosuccinimide (3.2 g, 18 mmol) followed by NH₄OAc (3.5 g, 45 mmol). The mixture was stirred for 3 hours, then water (30 mL) was added and the mixture was extracted with EtOAc (150 mL). The organic layer was washed with brine, dried over Na₂SO₄, filtered, concentrated in vacuo, and purified by column chromatography (5% EtOAc/heptane) to give 3.6 g of 11b as a yellow oil (yield 85%).

### C. 1-(Benzyloxy)-4-(benzyloxymethyl)-2-bromobenzene (11c, X=CH₂OBn)

Intermediate 11c was prepared following the general procedure of Aki et al., J Phys Chem, 2002, 106(14): 3436-3444.

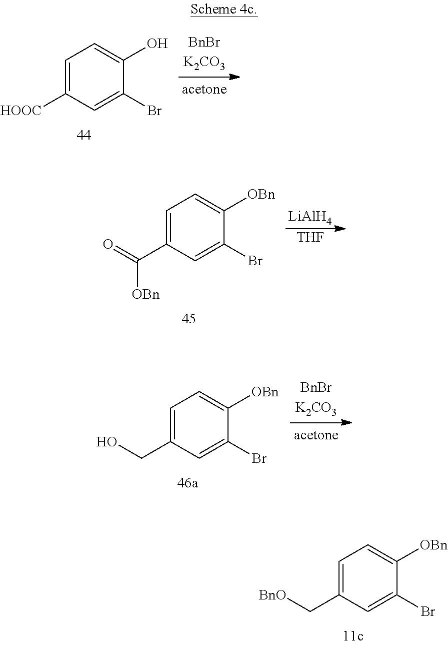

Step 1. Benzyl 4-(benzyloxy)-3-bromobenzoate (45). To a solution of 3-bromo-4-hydroxybenzoic acid 44 (10 g, 46 mmol) in acetone (200 mL) was added K₂CO₃ (20 g, 145 mmol) and benzyl bromide (12 mL, 101 mmol). The mixture was heated to reflux for 5 hours, then was cooled to room temperature. The solid was removed via filtration and the filtrate was concentrated in vacuo. The residue was dissolved in EtOAc (150 mL), and the solution was washed with brine, dried over Na₂SO₄, filtered, and concentrated in vacuo to yield a syrupy residue. The residue was recrystallized from 5% EtOAc/heptane to give 16.4 g of 45 as a white solid (yield 90%).

Step 2. (4-(Benzyloxy)-3-bromophenyl)methanol (46a). Benzyl 4-(benzyloxy)-3-bromobenzoate (45) (15 g, 37.8 mmol) was dissolved in 150 mL dry THF and cooled to 0° C. LiAlH₄ (2.9 g, 76 mmol) was added slowly in three portions. The mixture was stirred at room temperature for 12 hours, then was quenched by the addition of H₂O (3 mL), 15% NaOH (3 mL), and H₂O (7.6 mL). The precipitate was removed by filtration, and washed with EtOAc. The combined filtrates were concentrated to give a colorless oil which was purified by column chromatography (5%-10% EtOAc/heptane) to give 9.4 g of 46a as a colorless oil (yield 84%).

Step 3. 1-(Benzyloxy)-4-(benzyloxymethyl)-2-bromobenzene (11c, X=CH₂OBn). To a solution of (4-(benzyloxy)-3-bromophenyl)methanol (46a) (9 g, 30.7 mmol) in 100 mL acetone was added K₂CO₃ (12.7 g, 92 mmol) and benzyl bromide (4 mL, 33.8 mmol). The mixture was stirred for 5 hours, then was cooled to room temperature. The solid was removed by filtration and the filtrate was concentrated in vacuo to a residue which was purified by column chromatography (5% EtOAc/heptane) to give 10.6 g of 11c as a colorless oil (yield 90%).

### D. 1-(Benzyloxy)-4-(benzyloxy(methyl-d₂))-2-bromobenzene (11d, X=CH₂OBn)

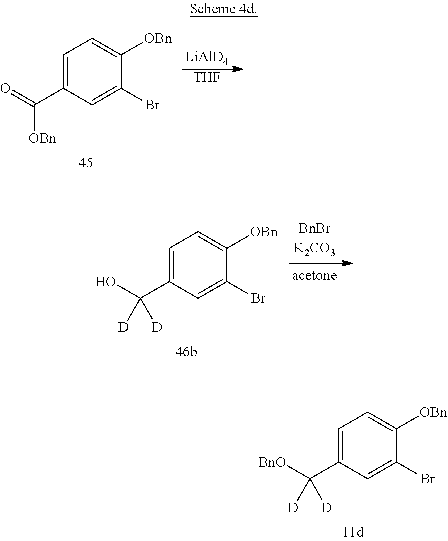

Step 1. (4-(Benzyloxy)-3-bromophenyl)-1,1-d₂-methanol (46b). To a solution of benzyl 4-(benzyloxy)-3-bromobenzoate (45) (15 g, 37.8 mmol) in 150 mL dry THF at 0° C. was added slowly in three portions LiAlD₄ (Cambridge Isotope, 99 atom % D) (3.1 g, 75.6 mmol). The mixture was stirred at room temperature for 12 hours, then was quenched by the addition of H₂O (3 mL), 15% NaOH (3 mL), and H₂O (7.6 mL). The precipitate was filtered and washed with EtOAc. The filtrate was concentrated in vacuo to give a colorless oil which was purified by column chromatography (5%-10% EtOAc/heptane) to give 9.0 g of 46b as a colorless oil (yield 81%).

Step 2. 1-(Benzyloxy)-4-(benzyloxy(methyl-d₂))-2-bromobenzene (11d, X=CH₂OBn). To a solution of (4-(benzyloxy)-3-bromophenyl)-1,1-d₂-methanol (46b) (9.0 g, 30.5 mmol) in 100 mL acetone was added K₂CO₃ (12.7 g, 92 mmol) and benzyl bromide (4 mL, 33.8 mmol). The mixture was stirred for 5 hours and cooled to room temperature. The solid was removed by filtration. The filtrate was concentrated in vacuo, and the residue was purified by column chromatography (5% EtOAc/heptane) to give 10.4 g of 11d as a colorless oil (yield 88%).

### Example 2

### Synthesis of (R)-2-(3-(Di(isopropyl-d₇)amino)-1-phenylpropyl)-4-methylphenol (Compound 106)

Compound 106 was prepared as outlined in Scheme 1 using appropriately deuterated reagents.

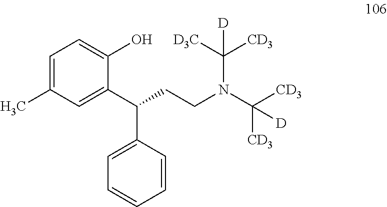

Step 1. (R)-3-(R)-3-(2-(Benzyloxy)-5-methylphenyl)-3-phenylpropanoyl)-4-phenyloxazolidin-2-one (12a, X=CH₃). To a solution of 11a (5 g, 18 mmol, see Example 1a) in THF (anhydrous, 20 mL) was added Mg (648 mg, 27 mmol). A small piece of iodine was added and the mixture was heated at 80° C. for 1 hour, then cooled to −50° C. CuBr-Me₂S (3.5 g, 9 mmol) was added. The mixture was stirred at −30° C. for 1 hour, then a solution of (R)-3-cinnamoyl-4-phenyloxazolidin-2-one (10) (6.3 g, 21.6 mmol, prepared as described by Andersson, P G et al., J Org Chem, 1998, 63(22): 8067 and Nicolas, E et al., J Org Chem, 1993, 58(3): 766-770) in THF (anhydrous, 40 mL) was added. The mixture was slowly warmed to room temperature then was stirred for another 6 hours, quenched with aqueous NH₄Cl (satd), and extracted with EtOAc (200 mL). The organic layer was washed with brine, dried over Na₂SO₄, filtered and concentrated in vacuo. The residue was purified by column chromatography (20% EtOAc/heptane) to give 7.1 g of 12a as a white solid (yield 80%).

Step 2. (R)-3-(2-(benzyloxy)-5-methylphenyl)-3-phenylpropanoic acid (13a, X=CH₃. To a solution of 12a (4 g, 8.1 mmol) in THF (30 mL) was added water (10 mL). The mixture was cooled to 0° C. and 30% H₂O₂ solution (7.2 mL, 64 mmol) was added followed by the addition of LiOH (388 mg, 16.2 mmol). The mixture was stirred at room temperature for 4 hours, acidified to pH=2 with 4N HCl, then was extracted with EtOAc (50 mL×3). The organic layer was dried over Na₂SO₄, filtered, concentrated in vacuo, and the residue was purified by column chromatography (20-50% EtOAc/heptane) to give 2.3 g of 13a as a white solid (yield 83%).

Step 3. (R)-3-(2-(Benzyloxy)-5-methylphenyl)-3-phenylpropanoyl chloride (14a, X=CH₃) To a solution of 13a (1.1 g, 2.9 mmol) in toluene (10 mL) was added thionyl chloride (1.1 mL, 15 mL) followed by the addition of 1 drop of dry pyridine. The mixture was heated to reflux for 1 hour. Thionyl chloride was removed under vacuum. Dry ether (20 mL) was added and the resulting solid was removed via filtration. The filtrate was concentrated in vacuo to give 1.1 g of crude 14a as a light-yellow solid.

Step 4. (R)-3-(2-(Benzyloxy)-5-methylphenyl)-N,N-di(isopropyl-d₇)-3-phenylpropanamide (15a, X=CH₃; R²=R³=CD(CD₃)₂). To a solution of 1.1 g of crude 14a in dry ether (20 mL) was added diisopropyl amine-d₁₄ [CDN Isotopes, 98 atom % D] (517 mg, 4.5 mmol) followed by Et₃N (1.25 mL, 9 mmol). The mixture was stirred for 4 hours, quenched by the addition of 2N HCl (15 mL), then extracted with EtOAc (50 mL). The organic layer was washed with aqueous NaHCO₃ (satd), brine, dried over Na₂SO₄, filtered, concentrated in vacuo, and the residue purified by column chromatography (30% EtOAc/heptane) to give 1.1 g of 15a as a colorless oil (yield 86%).

Step 5. (R)-3-(2-(Benzyloxy)-5-methylphenyl)-N,N-di(isopropyl-d₇)-3-phenylpropan-1-amine (16a, X=CH₃; R²=R³=CD(CD₃)₂1. To a solution of 15a (1.1 g, 2.48 mmol) in THF (anhydrous, 15 mL) at 0° C. was added LiAlH₄ (190 mg, 5.0 mmol). The mixture was stirred for 14 hours at room temperature, then quenched by the addition of H₂O (0.2 mL), 15% NaOH solution (0.2 mL), and H₂O (0.5 mL). The precipitate was filtered and washed with EtOAc. The filtrate was concentrated in vacuo to give 860 mg of 16a as a colorless oil (yield 80%).

Step 6. (R)-2-(3-(Di(isopropyl-d₇)amino)-1-phenylpropyl)-4-methylphenol L-tartrate (Compound 106 as its L-tartrate salt). To a solution of 16a (850 mg, 1.97 mmol) in methanol (20 mL) was added 10% Pd/C (2.1 g with 50% water, 1.0 mmol). The mixture was stirred under hydrogen (2 atm) for 10 hours, filtered through a Celite pad and the filtrate was concentrated in vacuo. The residue was purified by column chromatography (2% Et₃N in 5% MeOH/CH₂Cl₂) to give 570 mg of Compound 106 as its free base (yield 85%). The free base (570 mg, 1.68 mmol) was dissolved in 10 mL ethanol (absolute) followed by the addition of L-tartaric acid (277 mg, 1.85 mmol). The mixture was heated to reflux for 1 hour, then was kept in a refrigerator for 10 hours. The resulting salt was filtered and washed with cold ethanol to give 820 mg of Compound 106 as the L-tartrate salt. ¹H-NMR (300 MHz, DMSO-d₆): δ 2.17 (s, 3H), 2.28-2.33 (m, 2H), 2.67-2.75 (m, 2H), 3.96 (s, 2H), 4.30 (t, J=7.8, 1H), 6.67 (d, J=7.9, 1H), 6.80 (dd, J₁=8.2, J₂=1.8, 1H), 7.02 (d, J=2.0, 1H), 7.13-7.19 (m, 1H), 7.25-7.33 (m, 4H). ¹³C-NMR (75 MHz, DMSO-d₆): δ 21.02, 41.50, 45.67, 72.14, 115.73, 126.66, 128.00, 128.12, 128.50, 128.58, 128.87, 130.30, 144.74, 152.98, 174.69. HPLC (method: Waters Atlantis T3 2.1×50 mm 3 μm C18-RP column—gradient method 5-95% ACN+0.1% formic acid in 14 min (1.0 mL/min) with 4 min hold at 95% ACN; Wavelength: 254 nm): retention time: 4.88 min; 99+% purity. Chiral HPLC (method: 250 mm×4.6 mm Chiral OD column—isocratic method 95% hexane/5% isopropanol for 40 min (0.50 mL/min); Wavelength: 210 nm): retention time: 8.84 min (major enantiomer); 11.59 min (minor enantiomer); 99.4% ee purity. MS (M+H): 340.2. Elemental Analysis (C₂₆H₂₃D₁₄NO₇): Calculated: C=63.79; H=7.62; N=2.86. Found: C=63.79; H=7.55; N=2.86.

### Example 3

### Synthesis of (R)-2-(3-(Diisopropyl)amino)-1-phenylpropyl)-4-(methyl-d₃)phenol (Compound 112)

Compound 112 was prepared as outlined in Scheme 1 using appropriately deuterated reagents.

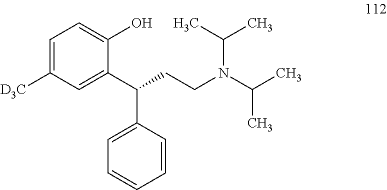

Step 1. (R)-3-(R)-3-(2-(Benzyloxy)-5-(methyl-d₃)phenyl)-3-phenylpropanoyl-1)-4-phenyloxazolidin-2-one (12b, X=CD₃. To a solution of 1-((2-bromo-4-(methyl-d₃)-phenoxy)methyl)benzene (11b) (3.5 g, 12.5 mmol, see Example 1b) in THF (anhydrous, 20 mL) was added Mg (450 mg, 18.8 mmol). A small piece of iodine was added and the mixture was heated at 80° C. for 1 hour, then was cooled to −50° C. CuBr-Me₂S (1.3 g, 6.3 mmol) was added. The mixture was stirred at −30° C. for 1 hour, then a solution of (R)-3-cinnamoyl-4-phenyloxazolidin-2-one (10) (5.5 g, 18.75 mmol, for reference see Example 2, step 1) in THF (anhydrous, 40 mL) was added. The mixture was slowly warmed to room temperature and stirred for another 6 hours, quenched by the addition of aqueous NH₄Cl (satd), and extracted with EtOAc (200 mL). The organic layer was washed with brine, dried over Na₂SO₄, filtered, and the filtrate was concentrated in vacuo. The residue was purified by column chromatography (20% EtOAc/heptane) to give 5.2 g of 12b as a white solid (yield 83%).

Step 2. (R)-3-(2-(Benzyloxy)-5-(methyl-d₃)-phenyl)-3-phenylpropanoic acid (13b, X=CD₃). To a solution of 12b (4 g, 8.1 mmol) in THF (30 mL) was added water (10 mL). The mixture was cooled to 0° C., and 30% H₂O₂ solution (7.2 mL, 64 mmol) was added followed by the addition of LiOH (388 mg, 16.2 mmol). The mixture was stirred at room temperature for 4 hours, acidified to pH=2 with 4N HCl, and extracted with EtOAc (50 mL×3). The organic extracts were combined and dried over Na₂SO₄, filtered, and the filtrate was concentrated in vacuo. The residue was purified by column chromatography (20-50% EtOAc/heptane) to give 2.3 g of 13b as a white solid (yield 83%).

Step 3. (R)-3-(2-(Benzyloxy)-5-(methyl-d₃)-phenyl)-3-phenylpropanoyl chloride (14b, X=CD₃). To 13b (1.2 g, 3.0 mmol) in toluene (10 mL) was added thionyl chloride (1.1 mL, 15 mL) followed by the addition of 1 drop of dry pyridine. The mixture was heated to reflux for 1 hour. Excess thionyl chloride was removed under vacuum. Dry ether (20 mL) was added and the resulting solid was removed via filtration. The filtrate was concentrated in vacuo to give 1.1 g of crude 14b as a light-yellow solid.

Step 4. (R)-3-(2-(Benzyloxy)-5-(methyl-d₃)-phenyl)-N,N-diisopropyl-3-phenylpropanamide (15b, X=CD₃, R²=R³=CH(CH₃)₂). To a solution of 1.1 g of crude 14b in dry ether (20 mL) was added diisopropyl amine (2.1 mL, 15 mmol). The reaction mixture was stirred for 4 hours, quenched by the addition of 2N HCl (15 mL), and extracted with EtOAc (50 mL). The organic layer was washed with aqueous NaHCO₃ (satd), brine, dried over Na₂SO₄, filtered, and the filtrate concentrated in vacuo. The residue was purified by column chromatography (30% EtOAc/heptane) to give 1.1 g of 15b as a colorless oil (yield 89%).

Step 5. (R)-3-(2-(Benzyloxy)-5-(methyl-d₃)-phenyl)-N,N-diisopropyl-3-phenylpropan-1-amine (16b, X=CD₃, R²=R³=CH(CH₃)₂). To a solution of 15b (1.1 g, 2.6 mmol) in THF (anhydrous, 15 mL) at 0° C. was added LiAlH₄ (210 mg, 5.2 mmol). The mixture was stirred for 14 hours at room temperature, then was quenched by the addition of H₂O (0.21 mL), 15% NaOH solution (0.21 mL), and H₂O (0.52 mL). The precipitate was filtered and washed with EtOAc. The filtrate was concentrated to give 880 mg of 16b as a colorless oil (yield 80%).

Step 6. 2-((R)-3-(Diisopropylamino)-1-phenylpropyl)-4-methylphenol L-tartrate (Compound 112 as its L-tartrate salt). To a solution of 16b (870 mg, 2.08 mmol) in methanol (20 mL) was added 10% Pd/C (2.1 g with 50% water, 1.0 mmol). The mixture was stirred under hydrogen (2 atm) for 10 hours then was filtered through a Celite pad and concentrated in vacuo. The residue was purified by column chromatography (2% Et₃N in 5% MeOH/CH₂Cl₂) to give 560 mg of Compound 112 as its free base (yield 81%). The free base (560 mg, 1.7 mmol) was dissolved in 10 mL ethanol (absolute) followed by the addition of L-tartaric acid (280 mg, 1.87 mmol). The mixture was heated to reflux for 1 hour, then was kept in a refrigerator for 10 h. The resulting salt was filtered and washed with cold ethanol to give 800 mg of Compound 112 as the L-tartrate salt. ¹H-NMR (300 MHz, DMSO-d₆): δ 1.08 (d, J=6.6, 12H), 2.15-2.17 (m, 0.6H), 2.26-2.32 (m, 2H), 2.65-2.73 (m, 2H), 3.37-3.45 (m, 2H), 3.98 (s, 2H), 4.30 (t, J=7.8, 1H), 6.66 (d, J=8.0, 1H), 6.80 (dd, J₁=8.2, J₂=2.2, 1H)), 7.01 (d, J=2.1, 1H), 7.13-7.19 (m, 1H), 7.24-7.33 (m, 4H). ¹³C-NMR (75 MHz, DMSO-d₆): δ 18.91, 33.71, 41.49, 45.60, 52.73, 72.18, 115.72, 126.63, 127.88, 128.09, 128.50, 128.58, 128.86, 130.37, 144.82, 152.98, 174.64. HPLC (method: Waters Atlantis T3 2.1×50 mm 3 μm C18-RP column—gradient method 5-95% ACN+0.1% formic acid in 14 min (1.0 mL/min) with 4 min hold at 95% ACN; Wavelength: 254 nm): retention time: 4.87 min; 99+% purity. Chiral HPLC (method: 250 mm×4.6 mm Chiral OD column—isocratic method 95% hexane/5% isopropanol for 40 min (0.50 mL/min); Wavelength: 210 nm): retention time: 8.88 min (major enantiomer); 11.62 min (minor enantiomer); 99.8% ee purity. MS (M+H): 329.1. Elemental Analysis (C₂₆H₃₄D₃NO₇): Calculated: C=62.25, H=7.79, N=2.93. Found: C=65.11, H=7.76, N=2.91.

### Example 4

### Synthesis of (R)-2-(3-(Di(isopropyl-d₇))amino)-1-phenylpropyl)-4-(methyl-d₃)phenol (Compound 100)

Compound 100 was prepared as outlined in Scheme 1 using appropriately deuterated reagents.

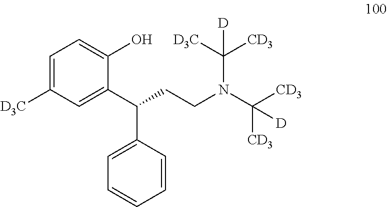

Step 1. (R)-3-(2-(Benzyloxy)-5-(methyl-d₃)-phenyl)-N,N-di(isopropyl-d₇)-3-phenylpropanamide (15e, X=CD₃, R²=R³=CD(CD₃)₂). To a solution of 1.1 g of crude 14b (see Example 3, step 3) in dry ether (20 mL) was added diisopropyl amine-d₁₄ [CDN Isotopes, 99 atom % D] (517 mg, 4.5 mmol) followed by Et₃N (1.25 mL, 9 mmol). The mixture was stirred for 4 hours, quenched by the addition of 2N HCl (15 mL), then extracted with EtOAc (50 mL). The organic layer was washed with aqueous NaHCO₃ (satd), brine, dried over Na₂SO₄, filtered and concentrated in vacuo. The residue was purified by column chromatography (30% EtOAc/heptane) to give 1.06 g of 15e as a colorless oil (yield 83%).

Step 2. (R)-3-(2-(Benzyloxy)-5-(methyl-d₃)-phenyl)-N,N-di(isopropyl-d₇)-3-phenylpropan-1-amine (16e, X=CD₃, R²=R³=CD(CD₃)₂). A solution of 15e (1.0 g, 2.47 mmol) was dissolved in THF (anhydrous, 15 mL) and cooled to 0° C. followed by the addition of LiAlH₄ (190 mg, 5.0 mmol). The mixture was stirred for 14 hours at room temperature, then quenched by the addition of H₂O (0.2 mL), 15% NaOH solution (0.2 mL), and H₂O (0.5 mL). The precipitate was filtered and washed with EtOAc. The filtrate was concentrated in vacuo to give 850 mg of 16e as a colorless oil (yield 79%).

Step 3. (R)-2-(3-(Di(isopropyl-d₇))amino)-1-phenylpropyl)-4-(methyl-d₃)phenol (Compound 100). To a solution of 16e (850 mg, 1.97 mmol) in methanol (20 mL) was added 10% Pd/C (2.1 g with 50% water, 1.0 mmol). The mixture was stirred under hydrogen (2 atm) for 10 hours then was filtered through a Celite pad. The filtrate was concentrated in vacuo and the residue was purified by column chromatography (2% Et₃N in 5% MeOH/CH₂Cl₂) to give 550 mg of Compound 100 as a free base (yield 80%). The free base (550 mg, 1.61 mmol) was dissolved in 10 mL ethanol (absolute) followed by the addition of L-tartaric acid (266 mg, 1.77 mmol). The mixture was heated to reflux for 1 hour then kept in a refrigerator for 10 h. The resulting salt was filtered and washed with cold ethanol to give 790 mg of Compound 100 as the L-tartrate salt. ¹H-NMR (300 MHz, DMSO-d₆): δ 1.04-1.07 (m, 0.7H), 2.16-2.18 (m, 0.7H), 2.26-2.28 (m, 2H), 2.63-2.75 (m, 2H), 3.43 (trace), 3.95 (s, 2H), 4.30 (t, J=7.8, 1H), 6.67 (d, J=7.9, 1H), 6.81 (dd, J₁=8.0, J₂=2.0, 1H)), 7.02 (d, J=1.8, 1H), 7.14-7.19 (m, 1H), 7.25-7.33 (m, 4H). ¹³C-NMR (75 MHz, DMSO-d₆): δ 41.48, 45.60, 72.02, 115.72, 126.65, 128.00, 128.11, 128.50, 128.58, 128.87, 130.31, 144.78, 152.97, 174.61. HPLC (method: Waters Atlantis T3 2.1×50 mm 3 μm C18-RP column—gradient method 5-95% ACN+0.1% formic acid in 14 min (1.0 mL/min) with 4 min hold at 95% ACN; Wavelength: 254 nm): retention time: 4.79 min; 99+% purity. Chiral HPLC (method: 250 mm×4.6 mm Chiral OD column—isocratic method 95% hexane/5% isopropanol for 40 min (0.50 mL/min); Wavelength: 210 nm): retention time: 8.87 min (major enantiomer); 11.61 min (minor enantiomer); 99.6% ee purity. MS (M+H): 343.2. Elemental Analysis (C₂₆H₂₀D₁₇NO₇): Calculated: C=63.40, H=7.57, N=2.84. Found: C=63.54, H=7.73, N=2.77.

### Example 5

### Synthesis of (R)-2-(3-(Di(isopropyl-d₇))amino)-1-phenylpropyl)-4-(hydroxymethyl)phenol (Compound 109)

Compound 109 was prepared as outlined in Scheme 1 using appropriately deuterated reagents.

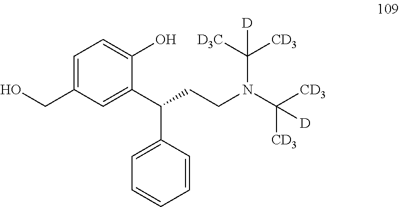

Step 1. (R)-3-((R)-3-(2-(Benzyloxy)-5-(benzyloxymethyl)phenyl)-3-phenylpropanoyl)-4-phenyloxazolidin-2-one (12c, X=CH₂OBn). To a solution of 11c (5 g, 13 mmol, see Example 1c) in THF (anhydrous, 20 mL) was added Mg (480 mg, 20 mmol). A small piece of iodine was added and the mixture was heated at 80° C. for 1 hour, and then cooled to −50° C. CuBr-Me₂S (1.4 g, 7 mmol) was added. The mixture was stirred at −30° C. for 1 hour, then a solution of (R)-3-cinnamoyl-4-phenyloxazolidin-2-one (10) (5.5 g, 19 mmol, for reference see Example 2, step 1) in THF (anhydrous, 40 mL) was added. The mixture was slowly warmed to room temperature and stirred for another 6 hours then was quenched by the addition of aqueous NH₄Cl (satd) and extracted with EtOAc (200 mL). The organic layer was washed with brine, dried over Na₂SO₄, filtered and concentrated in vacuo. The residue was purified by column chromatography (20% EtOAc/heptane) to give 6.0 g of 12c as a white solid (yield 77%).

Step 2. (R)-3-(2-(Benzyloxy)-5-(benzyloxymethyl)phenyl)-3-phenylpropanoic acid (13c, X=CH₂OBn). To a solution of 12c (4.8 g, 8.1 mmol) in THF (30 mL) was added water (10 mL). The mixture was cooled to 0° C. and 30% H₂O₂ solution (7.2 mL, 64 mmol) was added followed by the addition of LiOH (388 mg, 16.2 mmol). The mixture was stirred at room temperature for 4 hours, acidified to pH=2 with 4N HCl, and extracted with EtOAc (50 mL×3). The organic extracts were combined, dried over Na₂SO₄, filtered, and the filtrate was concentrated in vacuo. The residue was purified by column chromatography (20-50% EtOAc/heptane) to give 2.95 g of 13c as a white solid (yield 80%).

Step 3. (R)-3-(2-(Benzyloxy)-5-(benzyloxymethyl)phenyl)-3-phenylpropanoyl chloride (14c, X=CH₂OBn). To 13c (1.3 g, 2.9 mmol) in toluene (10 mL) was added thionyl chloride (1.1 mL, 15 mL) followed by the addition of 1 drop of dry pyridine. The mixture was heated to reflux for 1 hour. Thionyl chloride was removed under vacuum. Dry ether (20 mL) was added. The resulting solid was removed via filtration. The solution was concentrated in vacuo to give 1.3 g of crude 14c as a light-yellow solid.

Step 4. (R)-3-(2-(Benzyloxy)-5-(benzyloxymethyl)phenyl)-N,N-di(isopropyl-d₇)-3-phenylpropanamide (15c, X=CH₂OBn R₂=R₃=CD(CD₃)₂). To a solution of 1.3 g of crude 14c in dry ether (20 mL) was added diisopropyl amine-d₁₄ [CDN Isotope, 98 atom % D] (500 mg, 4.35 mmol) followed by the addition of Et₃N (1.25 mL, 9 mmol). The mixture was stirred for 4 hours, quenched by the addition of 2N HCl (15 mL) and extracted with EtOAc (50 mL). The organic layer was washed with aqueous NaHCO₃ (satd), brine, dried over Na₂SO₄, filtered, and the filtrate concentrated in vacuo. The residue was purified by column chromatography (30% EtOAc/heptane) to give 1.2 g of 15c as a colorless oil (yield 75%).

Step 5. (R)-3-(2-(Benzyloxy)-5-(benzyloxymethyl)phenyl)-N,N-di(isopropyl-d₇)-3-phenylpropan-1-amine (16c, X=CH₂OBn R₂=R₃=CD(CD₃)₂). A solution of 15c (1.2 g, 2.2 mmol) in THF (anhydrous, 15 mL) was cooled to 0° C. followed by the addition of LiAlH₄ (167 mg, 4.4 mmol). The mixture was stirred for 14 hours at room temperature, then quenched by the addition of H₂O (0.18 mL), 15% NaOH solution (0.18 mL), and H₂O (0.44 mL). The precipitate was filtered and washed with EtOAc. The filtrate was concentrated in vacuo and the residue purified by column chromatography (5% Et₃N in 50 EtOAc/heptane) to give 920 mg of 16c as a colorless oil (yield 78%).

Step 6. (R)-2-(3-(Di(isopropyl-d₇))amino)-1-phenylpropyl)-4-(hydroxymethyl)phenol (Compound 109). To a solution of 16c (910 mg, 1.7 mmol) in dichloromethane (20 mL) at −78° C. was added dropwise BCl₃ (5.9 mL in 1M dichloromethane, 5.9 mmol). The mixture was stirred for 3 hours then was quenched by the addition of H₂O and warmed to room temperature. The mixture was brought to pH=9 with aqueous NaHCO₃ (satd) and extracted with dichloromethane. The organic layer was dried over Na₂SO₄, filtered, and the filtrate concentrated in vacuo. The residue was purified by column chromatography (5% Et₃N in 5% methanol/dichloromethane) to give 440 mg of Compound 109 as a yellow oil (yield 72%).

### Example 6

### Synthesis of (R)-2-(3-(Di(isopropyl-d₇))amino)-1-phenylpropyl)-4-(hydroxymethyl)phenyl isobutyrate (Compound 117)

Compound 117 was prepared from Compound 109 as outlined in Scheme 1.

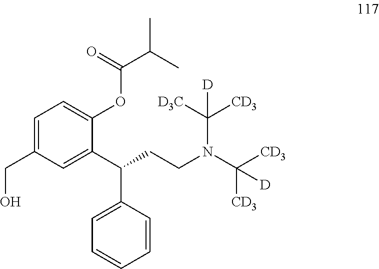

(R)-2-(3-(Di(isopropyl-d₇))amino)-1-phenylpropyl)-4-(hydroxymethyl)phenyl isobutyrate (Compound 117). To a solution of Compound 109 (440 mg, 1.24 mmol) in dichloromethane (10 mL) at −30° C. was added diisopropylethylamine (0.23 mL, 1.36 mmol) followed by the addition of isobutyryl chloride (0.13 mL, 1.24 mmol). The reaction mixture was stirred at −30° C. for 3 hours then was quenched by the addition of water and extracted with dichloromethane. The organic layer was dried over Na₂SO₄, filtered, and the filtrate concentrated in vacuo. The residue was purified by column chromatography (5% Et₃N in 20% EtOAc/heptane and then 5% Et₃N in 5% methanol/dichloromethane) to give 370 mg of Compound 117 as a yellow oil (yield 70%). ¹H-NMR (300 MHz, CDCl₃): δ 0.92 (m, 0.3H), 1.30 (d, J=11.1, 3H), 1.33 (d, J=11.1, 3H), 2.07-2.18 (m, 2H), 2.22-2.41 (m, 2H), 2.76-2.85 (m, 1H), 4.12 (t, J=7.4, 1H), 4.63 (s, 2H), 6.97 (d, J=8.2, 1H), 7.00-7.31 (m, 6H), 7.34 (d, J=1.5, 1H). ¹³C-NMR (75 MHz, CDCl₃): δ 18.97, 19.10, 34.20, 36.90, 41.77, 43.83, 64.90, 122.60, 125.60, 126.14, 126.89, 127.92, 128.33, 136.95, 138.64, 143.87, 147.94, 175.32. HPLC (method: 20 mm C18-RP column—gradient method 2-95% ACN+0.1% formic acid in 3.3 min with 1.7 min hold at 95% ACN; Wavelength: 210 nm): retention time: 2.89 min; 90% purity. MS (M+H): 426.3.

### Example 7

### Synthesis of (R)-2-(3-(Diisopropylamino)-1-phenylpropyl)-4-(hydroxyl(methyl-d₂))phenol (Compound 113)

Compound 113 was prepared as outlined in Scheme 1 using appropriately deuterated reagents.

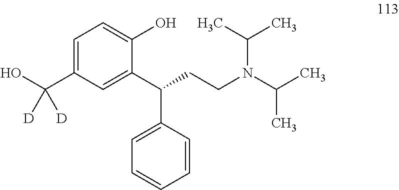

Step 1. (R)-3-((R)-3-(2-(Benzyloxy)-5-(benzyloxy(methyl-d₂))phenyl)-3-phenylpropanoyl)-4-phenyloxazolidin-2-one (12d, X=CD₂OBn). To a solution of 11d (5 g, 13 mmol, see Example 1d) in THF (anhydrous, 20 mL) was added Mg (480 mg, 20 mmol). A small piece of iodine was added and the mixture was heated at 80° C. for 1 hour, then was cooled to −50° C. CuBr-Me₂S (1.4 g, 7 mmol) was added. The mixture was stirred at −30° C. for 1 hour, then a solution of (R)-3-cinnamoyl-4-phenyloxazolidin-2-one (10) (5.5 g, 19 mmol, for reference see Example 2, step 1) in THF (anhydrous, 40 mL) was added. The mixture was slowly warmed to room temperature and stirred for another 6 hours, quenched by the addition of aqueous NH₄Cl (satd), and extracted with EtOAc (200 mL). The organic layer was washed with brine, dried over Na₂SO₄, filtered, and the filtrate was concentrated in vacuo. The residue was purified by column chromatography (20% EtOAc/heptane) to give 5.8 g of 12d as a white solid (yield 74%).

Step 2. (R)-3-(2-(Benzyloxy)-5-(benzyloxy(methyl-d₂))phenyl)-3-phenylpropanoic acid (13d, X=CD₂OBn). To a solution of 12d (5.0 g, 8.3 mmol) in THF (30 mL) was added water (10 mL). The mixture was cooled to 0° C., and 30% H₂O₂ solution (7.2 mL, 64 mmol) was added followed by the addition of LiOH (388 mg, 16.2 mmol). The mixture was stirred at room temperature for 4 hours, acidified to pH=2 with 4N HCl, and extracted with EtOAc (50 mL×3). The organic extracts were combined and dried over Na₂SO₄, filtered, and the filtrate concentrated in vacuo. The residue was purified by column chromatography (20-50% EtOAc/heptane) to give 2.82 g of 13d as a white solid (yield 75%).

Step 3. (R)-3-(2-(Benzyloxy)-5-(benzyloxy(methyl-d₂))phenyl)-3-phenylpropanoyl chloride (14d, X=CD₂OBn). To 13d (1.4 g, 3.1 mmol) in toluene (10 mL was added thionyl chloride (1.1 mL, 15 mmol) followed by the addition of 1 drop of dry pyridine. The mixture was heated to reflux for 1 hour. Excess thionyl chloride was removed under vacuum. Dry ether (20 mL) was added. The resulting solid was removed via filtration. The solution was concentrated to give 1.3 g of crude 14d as a light-yellow solid.

Step 4. (R)-3-(2-(Benzyloxy)-5-(benzyloxy(methyl-d₂))phenyl)-N,N-diisopropyl-3-phenylpropanamide (15d, X=CD₂OBn). To a solution of 14d in dry ether (20 mL) was added diisopropyl amine (2.1 mL, 15 mmol). The mixture was stirred for 4 hours, quenched by the addition of 2N HCl (15 mL) and extracted with EtOAc (50 mL). The organic layer was washed with NaHCO₃ (satd), brine, dried over Na₂SO₄, filtered, and the filtrate concentrated in vacuo. The residue was purified by column chromatography (30% EtOAc/heptane) to give 1.25 g of 15d as a colorless oil (yield 75%).

Step 5. (R)-3-(2-(Benzyloxy)-5-(benzyloxymethyl-d₂)phenyl)-N,N-diisopropyl-3-phenylpropan-1-amine (16d, X=CD₂OBn). To a solution of 15d (1.2 g, 2.2 mmol) in THF (anhydrous, 15 mL) at 0° C. was added LiAlH₄ (170 mg, 4.4 mmol). The mixture was stirred for 14 hours at room temperature, then quenched with H₂O (0.18 mL), 15% NaOH solution (0.18 mL), and H₂O (0.44 mL). The precipitate was filtered and washed with EtOAc. The organic layer was concentrated in vacuo and the residue purified by column chromatography (5% Et₃N in 50 EtOAc/heptane) to give 920 mg of 16d as a colorless oil (yield 80%).

Step 6. (R)-2-(3-(Diisopropylamino)-1-phenylpropyl)-4-(hydroxyl(methyl-d₂))phenol (Compound 113). To a solution of 16d (920 mg, 1.76 mmol) in dichloromethane (20 mL) at −78° C. was added dropwise BCl₃ (6.3 mL in 1M dichloromethane, 6.3 mmol). The mixture was stirred for 3 hours, then was quenched with H₂O and warmed to room temperature. The mixture was brought to pH=9 with NaHCO₃ (satd) and extracted with dichloromethane. The organic layer was dried over Na₂SO₄, filtered, and the filtrate concentrated in vacuo. The residue was purified by column chromatography (5% Et₃N in 5% methanol/dichloromethane) to give 425 mg of Compound 113 as a yellow oil (yield 70%).

### Example 8

### Synthesis of (R)-2-(3-(Diisopropylamino)-1-phenylpropyl)-4-(hydroxyl(methyl-d₂))phenyl isobutyrate (Compound 116)

Compound 116 was prepared from Compound 113 as outlined in Scheme 1.

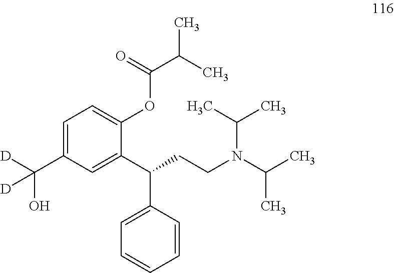

(R)-2-(3-(Diisopropylamino)-1-phenylpropyl)-4-(hydroxyl(methyl-d₂))phenyl isobutyrate (Compound 116). A solution of Compound 113 (420 mg, 1.22 mmol) in dichloromethane (10 mL) was cooled to −30° C., then diisopropylethylamine (0.23 mL, 1.35 mmol) was added followed by the addition of isobutyryl chloride (0.13 mL, 1.23 mmol). The mixture was stirred at −30° C. for 3 hours, then was quenched with water, extracted with dichloromethane, dried over Na₂SO₄, filtered, and the filtrate concentrated in vacuo. The residue was purified by column chromatography (5% Et₃N in 20% EtOAc/heptane and then 5% Et₃N in 5% methanol/dichloromethane) to give 360 mg of Compound 116 as a yellow oil (yield 70%). ¹H-NMR (300 MHz, CDCl₃): δ 0.91 (d, J=6.4, 12H), 1.30 (d, J=11.1, 3H), 1.32 (d, J=11.1, 3H), 2.08-2.17 (m, 2H), 2.30-2.37 (m, 2H), 2.76-2.85 (m, 1H), 2.91-3.00 (m, 2H), 4.11 (t, J=7.6, 1H), 6.96 (d, J=8.2, 1H), 7.12-7.32 (m, 6H), 7.34 (d, J=2.3, 1H). ¹³C-NMR (75 MHz, CDCl₃): δ 18.98, 19.10, 20.59, 34.20, 36.93, 41.77, 43.82, 48.71, 122.62, 125.65, 126.14, 126.95, 127.92, 128.34, 136.98, 138.48, 143.86, 147.98, 175.32. HPLC (method: 20 mm C18-RP column—gradient method 2-95% ACN+0.1% formic acid in 3.3 min with 1.7 min hold at 95% ACN; Wavelength: 210 nm): retention time: 2.85 min; 90% purity. MS (M+H): 414.3.

### Example 9

### Synthesis of (R)-2-(3-(Di(isopropyl-d₇)amino)-1-phenylpropyl)-4-(hydroxyl(methyl-d₂))phenol (Compound 103)

Compound 103 was prepared as outlined in Scheme 1 using appropriately deuterated reagents.

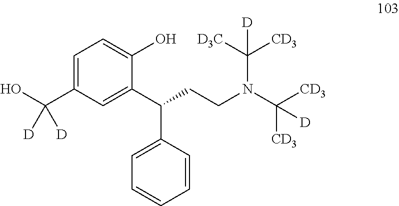

Step 1. (R)-3-(2-(Benzyloxy)-5-(benzyloxy(methyl-d₂))phenyl)-N,N-di(isopropyl-d₇)-3-phenylpropanamide (15f, X=CD₂OBn, R²=R³=CD(CD₃)₂). To a solution of 1.3 g of crude 14d (see Example 7, Step 3) in dry ether (20 mL) was added diisopropyl amine-d₁₄ [CDN Isotope, 98 atom % D] (500 mg, 4.35 mmol) followed by the addition of Et₃N (1.25 mL, 9 mmol). The mixture was stirred for 4 hours, quenched with 2N HCl (15 mL), and extracted with EtOAc (50 mL). The organic layer was washed with aqueous NaHCO₃ (satd), brine, dried over Na₂SO₄, filtered, and the filtrate concentrated in vacuo. The residue was purified by column chromatography (30% EtOAc/heptane) to give 1.26 g of 15f as a colorless oil (yield 74%).

Step 2. (R)-3-(2-(Benzyloxy)-5-(benzyloxy(methyl-d₂))phenyl)-N,N-di(isopropyl-d₇)-3-phenylpropan-1-amine (16f, X=CD₂OBn, R²=R³=CD(CD₃)₂). To a solution of 15f (1.2 g, 2.18 mmol) in THF (anhydrous, 15 mL) at 0° C. was added LiAlH₄ (170 mg, 4.4 mmol). The mixture was stirred for 14 hours at room temperature, then quenched with H₂O (0.18 mL), 15% NaOH solution (0.18 mL), and H₂O (0.44 mL). The precipitate was filtered and washed with EtOAc. The filtrate was concentrated in vacuo and the residue purified by column chromatography (5% Et₃N in 50 EtOAc/heptane) to give 960 mg of 16f as a colorless oil (yield 82%).

Step 3. (R)-2-(3-(Di(isopropyl-d₇)-amino)-1-phenylpropyl)-4-(hydroxyl(methyl-d₂))phenol (Compound 103). To a solution of 16f (960 mg, 1.79 mmol) in dichloromethane (20 mL) at −78° C. was added dropwise BCl₃ (6.3 mL in 1M dichloromethane, 6.3 mmol). The mixture was stirred for 3 hours, then was quenched with H₂O and warmed to room temperature. The mixture was brought to pH=9 with aqueous NaHCO₃ (satd), and extracted with dichloromethane. The organic layer was dried over Na₂SO₄, filtered, and the filtrate concentrated in vacuo. The residue was purified by column chromatography (5% Et₃N in 5% methanol/dichloromethane) to give 460 mg of Compound 103 as a yellow oil (yield 73%).

### Example 10

### Synthesis of (R)-2-(3-(Di(isopropyl-d₇)amino)-1-phenylpropyl)-4-(hydroxyl(methyl-d₂))phenyl isobutyrate (Compound 118)

Compound 118 was prepared from Compound 103 as outlined in Scheme 1.

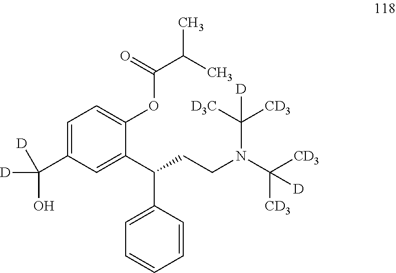

(R)-2-(3-(Di(isopropyl-d₇)amino)-1-phenylpropyl)-4-(hydroxyl(methyl-d₂))phenyl isobutyrate (Compound 118). To a solution of Compound 103 (460 mg, 1.29 mmol) in dichloromethane (10 mL) at −30° C. was added diisopropylethylamine (0.25 mL, 1.42 mmol) followed by the addition of isobutyryl chloride (0.14 mL, 1.29 mmol). The mixture was stirred at −30° C. for 3 hours, then was quenched with water, extracted with dichloromethane, dried over Na₂SO₄, filtered, and the filtrate concentrated in vacuo. The residue was purified by column chromatography (5% Et₃N in 20% EtOAc/heptane and then 5% Et₃N in 5% methanol/dichloromethane) to give 385 mg of Compound 118 as a yellow oil (yield 70%). ¹H-NMR (300 MHz, CDCl₃): δ 0.90 (trace), 1.30 (d, J=11.1, 3H), 1.33 (d, J=11.1, 3H), 2.13-2.17 (m, 2H), 2.30-2.36 (m, 2H), 2.76-2.85 (m, 1H), 4.11 (t, J=7.6, 1H), 6.97 (d, J=8.2, 1H), 7.13-7.33 (m, 6H), 7.34 (d, J=1.5, 1H). ¹³C-NMR (75 MHz, CDCl₃): δ 18.98, 19.10, 34.20, 36.87, 41.76, 43.83, 122.62, 125.67, 126.15, 126.96, 127.91, 128.34, 136.98, 138.48, 143.85, 147.99, 175.32. HPLC (method: 20 mm C18-RP column—gradient method 2-95% ACN+0.1% formic acid in 3.3 min with 1.7 min hold at 95% ACN; Wavelength: 210 nm): retention time: 2.87 min; 90% purity. MS (M+H): 428.4.

### Example 11

### Evaluation of Metabolic Stability in Human Liver Microsomes

Materials: Human liver microsomes (20 mg/mL) were obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) were purchased from Sigma-Aldrich.

Stock solutions (7.5 mM) of Compound 100, Compound 106, Compound 112 and tolterodine were separately prepared in DMSO. The 7.5 mM stock solutions were diluted to 50 μM in acetonitrile (ACN). The 20 mg/mL human liver microsomes were diluted to 0.625 mg/mL in 0.1 M potassium phosphate buffer, pH 7.4, containing 3 mM MgCl₂. The diluted microsomes were added to wells of a 96-well deep-well polypropylene plate in duplicate. Ten μL of one of the 50 μL test compound solutions was added to the microsomes and the mixture was pre-warmed for 10 minutes. Reactions were initiated by addition of pre-warmed NADPH solution. The final reaction volume was 0.5 mL and contained 0.5 mg/mL human liver microsomes, 1 μM test compound, and 2 mM NADPH in 0.1 M potassium phosphate buffer, pH 7.4, and 3 mM MgCl₂. The reaction mixtures were incubated at 37° C., and 50 μL aliquots were removed at 0, 5, 10, 20, and 30 minutes and added to shallow-well 96-well plates which contained 50 μL of ice-cold ACN with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer. 7-ethoxy coumarin (1 μM) was used as a positive control. The experiment was repeated as second time to confirm the results.

Data analysis: The in vitro t_{1/2}s for test compounds were calculated from the slopes of the linear regression of % parent remaining (ln) vs incubation time relationship using the following formula: in vitro t_{1/2}=0.693/k, where k=−[slope of linear regression of % parent remaining (ln) vs incubation time]. Data analysis was performed using Microsoft Excel Software.

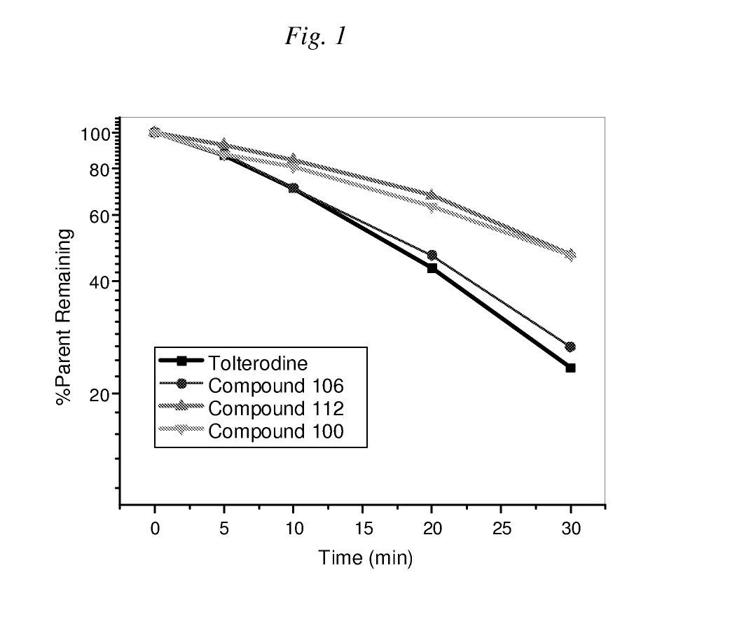

The results are shown in FIG. 1 and Table 2, below.

TABLE 2
Calculated Half-Lives of Compounds of the Invention in Human
Liver Microsomes.

| | t_{1/2} (minutes) | | | % |
|---|---|---|---|---|
| Compound | Experiment 1 | Experiment 2 | Ave t_{1/2} | Difference* |
| Tolterodine | 13.4 | 14.2 | 13.8 | — |
| Compound 100 | 23.5 | 28.0 | 25.8 | 87.0 |
| Compound 106 | 14.6 | 15.6 | 15.1 | 9.4 |
| Compound 112 | 24.3 | 28.9 | 26.6 | 92.8 |

*% Difference = [(deuterated species) − (nondeuterated species)](100)/(nondeuterated species)

Compounds 100 and 112 demonstrate a substantially increased half-life in human liver microsomes as compared to tolterodine. Surprisingly, Compound 106, which contains 14 deuterium atoms, showed little change in stability as compared to tolterodine. In contrast, Compound 112, which only contains three deuterium atoms demonstrated an increase in stability. The time course of metabolism is consistent with these results (FIG. 1).

Without being bound by theory, applicants believe that deuteration of the 5-methyl group on the phenyl moiety leads to increased stability of the compounds of this invention that contain a 2-hydroxyl on that phenyl moiety.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula II: 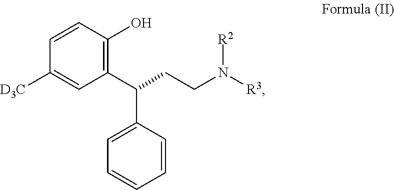
or a pharmaceutically acceptable salt thereof, wherein:
R² and R³ are each, independently, an isopropyl group bearing zero to seven deuterium atoms.

2. The compound of claim 1, wherein each of R² and R³ are independently selected from —CD(CD₃)₂ and —CH(CH₃)₂.

3. The compound of claim 2 selected from Compound 100 or Compound 112.

4. The compound of claim 1, wherein any atom not designated as deuterium is present at its natural isotopic abundance.

5. A pyrogen-free pharmaceutical composition comprising an effective amount of a compound of claim 1; and a pharmaceutically acceptable carrier.

6. The composition of claim 5 further comprising an effective amount of a second therapeutic agent useful in treating a patient suffering from or susceptible to a disease or condition selected from unstable or overactive bladder, incontinence, infection, lower urinary tract disorders, memory or cognition impairment, heart failure, pneumonia, benign prostatic hyperplasia, prostatic hypertrophy, respiratory disorders, asthma, female sexual dysfunction, or cystitis.

7. The composition of claim 6, wherein said second therapeutic agent is selected from an alpha-adrenergic receptor antagonist; a bicifadine compound; a statin; a dehydroepiandrosterone (DHEA) congener; an alpha-2-delta subunit calcium channel modifier; a selective serotonin reuptake inhibitor (SSRI) or a selective norepinephrine uptake inhibitor; an androgen; an estrogen; an estrogen agonist; an EGF receptor antagonist; a 5HT1a receptor modifier; a thienopyranecarboxamide derivative; and a 5a reductase inhibitor.

8. The composition of claim 7, wherein the second therapeutic agent is tamsulosin.

9. A method for treating a patient subject suffering from, or susceptible to, a disease or condition selected from unstable or overactive bladder, incontinence, infection, lower urinary tract disorders, memory or cognition impairment, heart failure, pneumonia, benign prostatic hyperplasia, prostatic hypertrophy, respiratory disorders, asthma, female sexual dysfunction, or cystitis comprising the step of administering to the patient in need thereof a composition of claim 5.

10. The method of claim 9, wherein the disease or condition is selected from overactive bladder and incontinence.

11. The method of claim 9 comprising the additional step of co-administering to the patient in need thereof a second therapeutic agent is:
a. selected from an alpha-adrenergic receptor antagonist, a bicifadine compound, an alpha-2-delta subunit calcium channel modifier, a selective serotonin reuptake inhibitor (SSRI), a selective norepinephrine uptake inhibitor, a 5HT1a receptor modifier, a thienopyranecarboxamide derivative, and a 5a reductase inhibitor; wherein the patient is suffering from or susceptible to a lower urinary tract disorder;
b. a dehydroepiandrosterone (DHEA) congener; wherein the patient is suffering from or susceptible to inflammation;
c. a statin, wherein the patient is suffering from or susceptible to a respiratory disease;
d. selected from an androgen, an estrogen or an estrogen agonist; and the patient is suffering from or susceptible to female sexual dysfunction; or
e. selected from an EGF receptor antagonist, or a thienopyranecarboxamide derivative; and the patient is suffering from or susceptible to benign prostatic hyperplasia, or prostatic hypertrophy.

12. The method of claim 11, wherein the second therapeutic agent is tamsulosin and the patient is suffering from or susceptible to benign prostatic hyperplasia, or prostatic hypertrophy.
